(19) United States
(12) Patent Application Publication
(10) Pub. No.: US 2023/0158272 A1
(43) Pub. Date: May 25, 2023

(54) SYSTEMS FOR PROVIDING AROMATHERAPY AND DEVICES INCORPORATED THEREIN FOR DIFFUSING ESSENTIAL OILS

(71) Applicant: YUSUF RANSOME, NEW HAVEN, CT (US)

(72) Inventor: YUSUF RANSOME, NEW HAVEN, CT (US)

(21) Appl. No.: 17/993,041

(22) Filed: Nov. 23, 2022

Related U.S. Application Data
(60) Provisional application No. 63/282,793, filed on Nov. 24, 2021.

(51) Int. Cl. A61M 21/02; H04M 1/21; B05B 1/24

(52) U.S. Cl. CPC A61M 21/02; H04M 1/21; B05B 1/24; A61M 2021/0016

(57) ABSTRACT

A system for providing aromatherapy to a user is described herein. The system comprises a mobile computing device including a mobile application to control the system; a diffuser mounted to the mobile computing device, the diffuser including: a body, one or more cavities in the body, one or more heating elements positioned to provide localized heating to the one or more cavities, a processor, and one or more non-transitory computer storage mediums. The system also includes a plurality of carriers including scented material, the carriers sized and configured to reversibly fill a cavity and position scented material proximate a heating element. The non-transitory computer storage mediums are encoded with one or more computer programs that, when executed by the processor in response to instructions from the mobile computing device, instruct the heating elements to apply heat from the heating elements to activate the scented material and deliver a predetermined aromatherapy experience to the user. Corresponding methods also are disclosed.

### RELATED APPLICATIONS

This application claims the benefit of U.S. Provisional Application No. 63/282,793 filed on Nov. 24, 2021, the contents of which are incorporated by reference herein in their entirety.

### BACKGROUND

This disclosure relates generally to an aromatherapy device and more particularly to a portable aromatherapy device configured for use in stress management and wellness promotion.

### SUMMARY

Aspects of the present disclosure are directed to a system for providing aromatherapy to a user that can be controlled via the user's mobile phone. The system includes an electronic-powered diffuser that can be connected to and controlled by the user directly via the device as well as via the user's mobile phone (or other device such as an Ipad or tablet computer) through a complementary mobile application (app) that helps the user decide on the length of diffusion, and other inputs (e.g., specific scent). The mobile app will store usage data to a cloud and aggregated user information will be used with artificial intelligence algorithms to recommend future experiences. Scented materials inserted into the diffuser can be activated to deliver combinations of scents to a user and thus facilitate predetermined aromatherapy experiences.

Another embodiment disclosed herein is system for providing aromatherapy to a user, the system comprising a mobile computing device including a mobile application to control the system, and a diffuser mounted to the mobile computing device. The diffuser includes a body, one or more cavities in the body, one or more heating elements positioned to provide localized heating to the one or more cavities, a processor, and one or more non-transitory computer storage mediums. The system also includes a plurality of carriers including scented material, the carriers sized and configured to reversibly fill a cavity and position scented material proximate a heating element. The non-transitory computer storage mediums are encoded with one or more computer programs that, when executed by the processor in response to instructions from the mobile computing device, instruct the heating elements to apply heat from the heating elements to activate the scented material and deliver a predetermined aromatherapy experience to the user.

Another embodiment is a method comprising obtaining a system for providing aromatherapy to a user, the system compromising of a body, one or more cavities in the body, one or more heating elements positioned to provide localized heating the one or more cavities, a processor, and one or more non-transitory computer storage mediums, inserting a scented component in at least one cavity of the body, and activating the heating element using a computing device to diffuse scented material.

### BRIEF DESCRIPTION OF THE DRAWINGS

FIG. 1 shows a top view of a first embodiment with the cover partially off.

FIG. 2A depicts a side view of the device's opened case.

FIG. 2B shows the inside of the case lid.

FIG. 3 depicts a top view of an exemplary embodiment of the circuit.

FIG. 4A is a top view of the circuit, removed from the case, without the heat sink.

FIG. 4B is a top view of the circuit, removed from the case, with the heat sink.

FIG. 5 depicts the heat sink, pad-side up.

FIG. 6 depicts the enabled app screen.

FIG. 7 depicts a second embodiment of the device attached to a mobile device.

FIG. 8 depicts a front view of the second embodiment.

FIG. 9 depicts a custom set of varied aromatherapy tablets.

FIG. 10 depicts a custom set of one type of aromatherapy tablet.

FIG. 11 depicts the enabled app screen on a mobile device.

### DETAILED DESCRIPTION

The apparatus described herein is a portable aromatherapy device which can attach to a mobile device and can provide relaxing aromatherapy to its user at any location. The technology is designed as a stress management system as well as to improve one's wellness experience. For example, when used for stress management, if a person experiences an acute stress event on suddenly, the portability advantage of this technology is that it allows the user to obtain aromatherapy through the device so that they can regulate the effects of stress at that instant, thereby mitigating the likelihood that the effects of that stress event becomes cumulative or has an adverse consequence on a behavior or future health outcome. When used for wellness optimization, a user may use the technology in complement with other wellness practices to augment their experience and also use the technology to promote relaxation and optimal performance of their neurological and cardiovascular systems. The device is configured to accommodate a variety of essential oils and scented materials that target specific mechanistic pathways to health (e.g., lavender scent to promote relaxation)

In some embodiments, the device is sufficiently small that is can be attached to a smartphone. In embodiments, essential oils are diffused by heating up an absorbent pad soaked with the essential oil. The device is controlled by a smartphone-enabled computerized application, which can be configured for Android, iOS and other operating systems. In some cases, all of the power for heating the oil comes from the phone's charging port, such as through a micro USB cable or another type of electronic hardwire or wireless power system. In some cases the device also is configured to be powered from a USB cable through an independent power sources such as a wall outlet or portable battery. In some embodiments, the device attaches to the back of the smartphone in a sleek manner, minimizing bulk and easing the portability of the product.

As used herein, the term “diffuser” means a device that produces a fine mist of liquid.

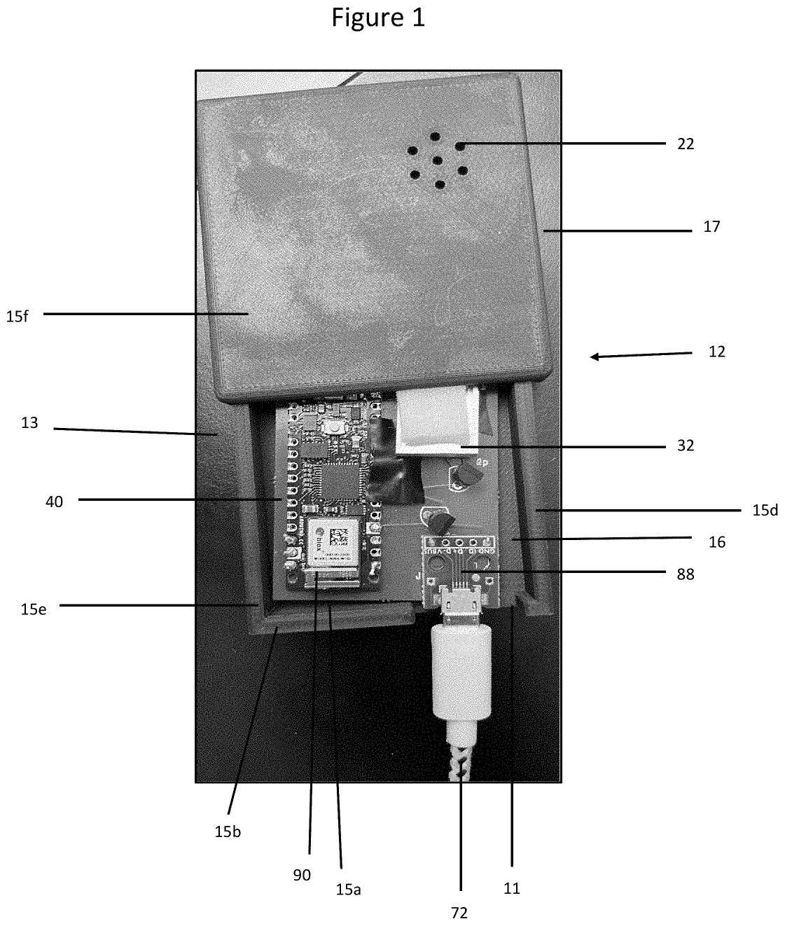

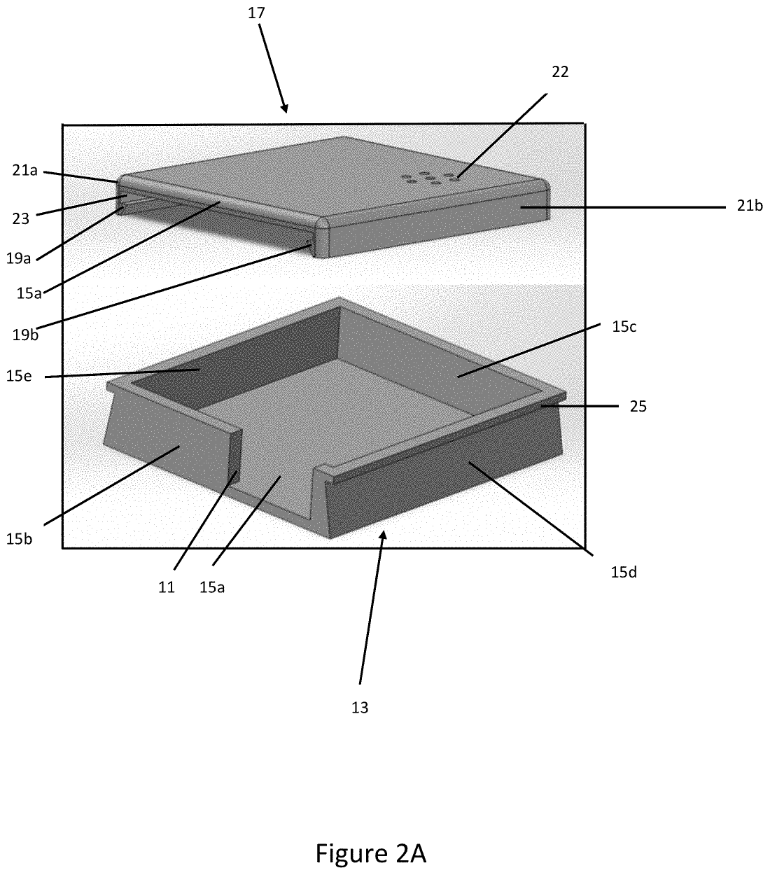

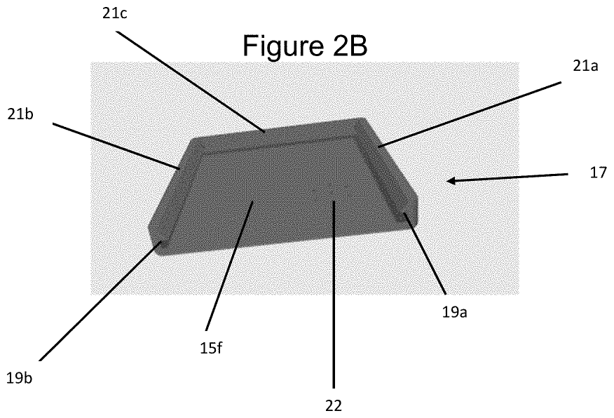

Referring to the drawings, FIG. 1 shows an embodiment of an aromatherapy system 10, where the case or body 12 is open and the circuit board 40 is shown. In the embodiment shown in FIGS. 1-2, the body is rectangular an includes bottom portion 13 with a lower wall 15a that supports internal components, opposite first and second side walls 15b and 15c, respectively, and opposite third and fourth side walls 15d and 15e, respectively. The first side wall 15b includes at least one opening 11 which is made accessible for the placement of a USB device. When the body 12 is closed, a lid 17 includes a planar top section that forms the upper wall 15f which is opposite to the lower wall 15a. The lid 17 also has a left side wall 21a, a right side wall 21b and a rear side wall 21c. The front side of the lid 17 is open. It is noted that the opening 11 can be formed in any of the side walls of the body, or in the upper wall 15f or the lower wall 15a depending upon the arrangement of components inside the body 12. When the body 12 is closed, an upper slot 23 formed between the inner side of the upper wall 15f, the inner sides of walls 21a, 21b, and above a pair of lips 19a and 19b formed on the inner sides of walls 21a and 21b, has a size and shape suitable to receive an outwardly extending rib 25 formed along the upper edges of second, third and fourth side walls 15c, 15d and 15e, respectively.

The body 12 is formed from a material that is sufficiently durable and rigid to support the internal components and remains rigid when heated to the temperatures to which the scented material 50 is heated. Some versions of the body 12 comprise a thermoplastic polymer or a thermoset polymer, which optionally may be reinforced with a filler. One non-limiting example of a suitable material is carbon reinforced thermoplastic polymer, such as carbon-reinforced nylon. In some embodiments, the bottom portion of the body measures 55-65 mm by 60-65 mm by 8-15 mm. In embodiments, the dimensions of the body 12 are about 60 mm by about 63 mm by about 10 mm. In embodiments, the body 12 is formed by molding, such as by injection molding or compression molding.

Referring to FIGS. 2A and 2B, the body 12 is shown from a side view, made up of the lid 17 and bottom portion 13 separated. In the illustrated embodiment, the lid 17 has a set of lips 19 on the inside of each side extending along the length of the case from first side wall 15a of the lid 17 to the second side wall 15b to hold the scented material above the circuit. In some embodiments, the lid 17 has one or more breathing holes 22 above the heating sink 32, to maintain the temperature of the device and allow for ventilation.

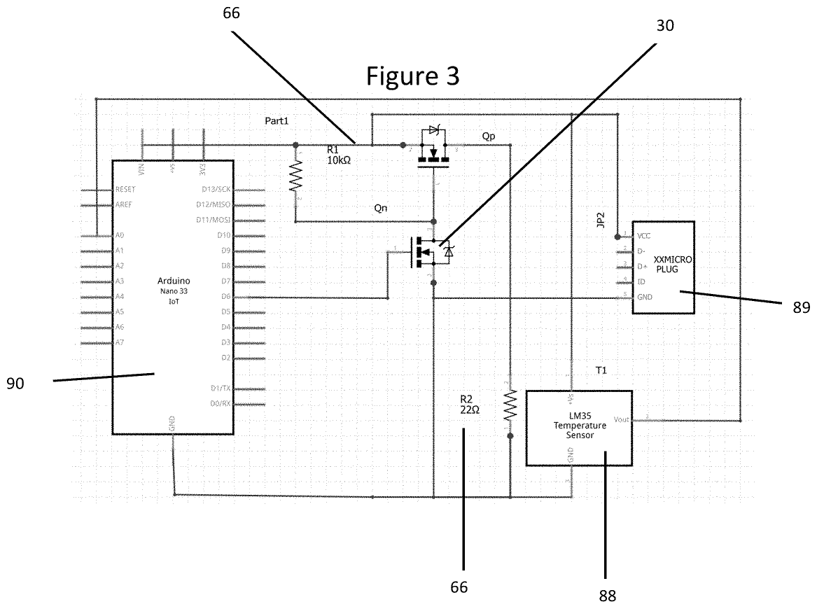

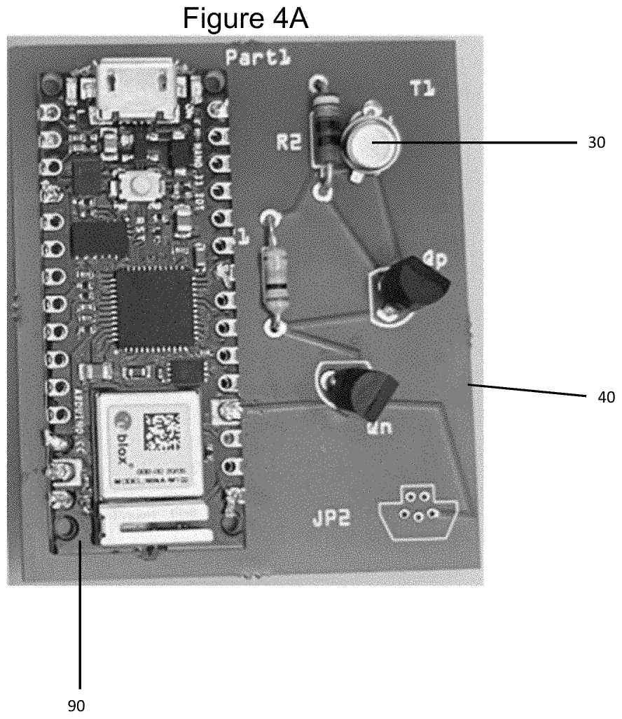

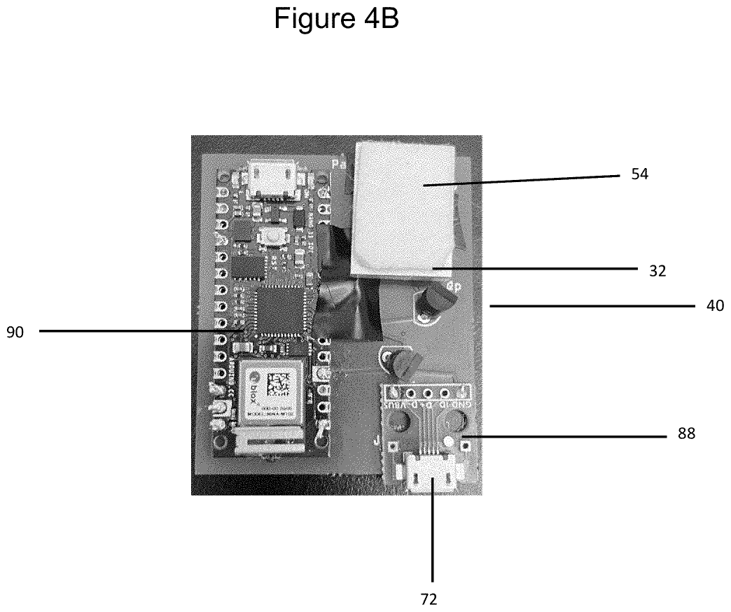

FIG. 3 schematically shows the circuitry for one of the embodiments disclosed herein. The circuit includes a microcontroller 90 (Arduino) that is electronically connected to a temperature sensor 88, a heating element 30 and a plug 89. FIGS. 4A-4B show non-limiting examples of microcontroller 90, heating element 30, heat sink 32, temperature sensor 88 and plug 89.

Referring to FIGS. 3, 4A and 4B, when installed in the cavities 16, the scented material 50 is positioned proximate one or more heating elements 30. More specifically, the heat sink 32 is positioned on top of the heating elements 30 and the scented pad 54 (or other scented component) is placed on top of the heat sink 32. The lid 17 is then placed on the body 12. When the heating elements 30 are activated, heat is likewise applied to the scented material 50, activating the scented material 50 and releasing a scent from the diffuser 20. In some embodiments, two or more scented materials 50 can be provided to the diffuser 20 at the same time. In some embodiments, scented materials 50 in the diffuser 20 can be activated alone, in series with other scents, in combination with other scents, or combinations thereof. In some embodiments, the scented materials 50 are interchangeable, e.g., scented materials 50 can be replaced with other scented materials 50 when desired by the user, e.g., when a different experience is desired, when a scented material 50 is exhausted and no longer producing scent, etc.

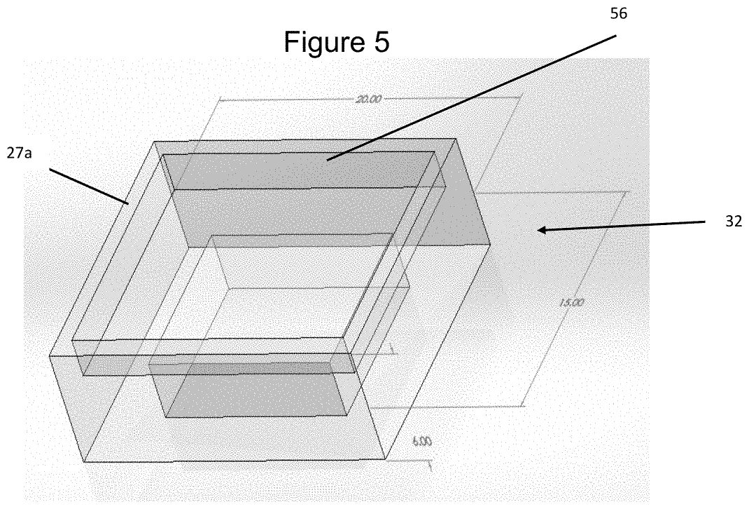

Referring to FIG. 5, in a non-limiting exemplary embodiment of the heat sink 32, it is shown with the absorbent pad-side up 56. Both sides of the heat sink 32 have lips 27a, 27b. The shallower lip 27a on the pad 56 side ensures no oil spills over the side of the heat sink 32, and the deeper lip 27b on the resistor 62 side provides a space to contact the resistor 62 and temperature sensor 88, which was then filled with a thermal adhesive.

Referring again to FIG. 5, in some embodiments, the heat sink 32 is created from a material having high thermal conductivity, non-limiting examples of which include aluminum, copper, graphite, iron, nickel, tungsten, and combinations thereof. The heat sink 32 was secured to a resistor 62 of 15-30-Ω and to a temperature sensor 88 configured to sense temperatures in a range of at least about 40 degrees C. to about 60 degrees C. with an accuracy of about 0.5 degrees, or 0.3 degrees, or less by a thermal adhesive. The thermal adhesive is configured to provide for an efficient means for the resistor 62 to transfer as much of its energy as possible into the heat sink 32, and then into the oil-soaked pad 56. The oil-absorbing pad 56 is formed from a highly oil-absorbent material, for example a plant fiber such as cellulose, or from wool, or an absorbent synthetic material such as polyester, or a blend of polyester and rayon or nylon.

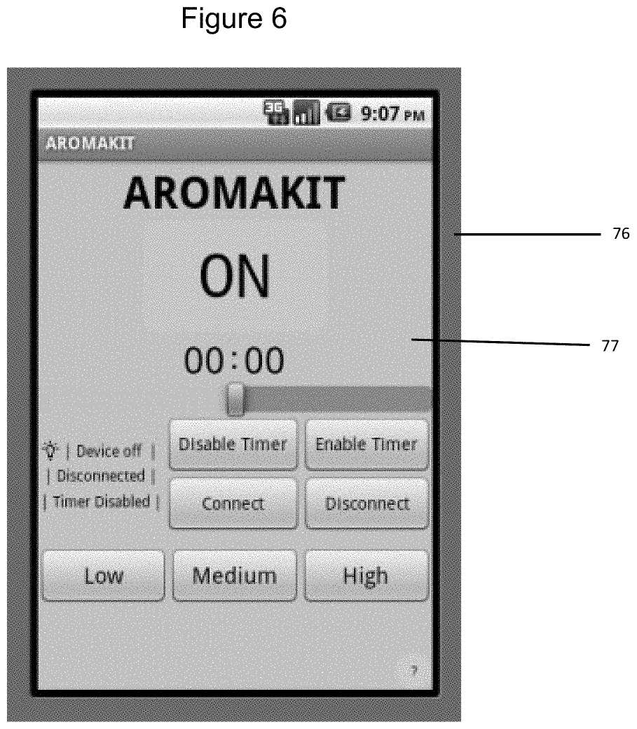

FIG. 6 shows a screen shot of the app 77 on a mobile device 76. The illustrated screen enables a user to start heating the scented material 50. The user has the option of setting a timer that will result in the heater 30 being automatically shut off after use. The use also has the option to use a low medium or high setting for the rate at which the scented material 50 is diffused. In some embodiments, the system 10 includes a status module to identify and provide status information related to the system, e.g., battery level, the identity of the scent material(s) present in the system, the amount of scent material present in the system 10, errors in the hardware 68 or software 70 of the system, etc.

Referring to FIG. 6, these exemplary embodiments are the designs used for the device which allows a user to input a desired output. The embodiment includes two Unique Identifiers (“UIDs) 82. One Unique Identifier 82 is a service UID which identifies the specific Bluetooth Low Energy device to connect to. The second UID 82 is a characteristic UID which indicates specific values for the device to read or change.

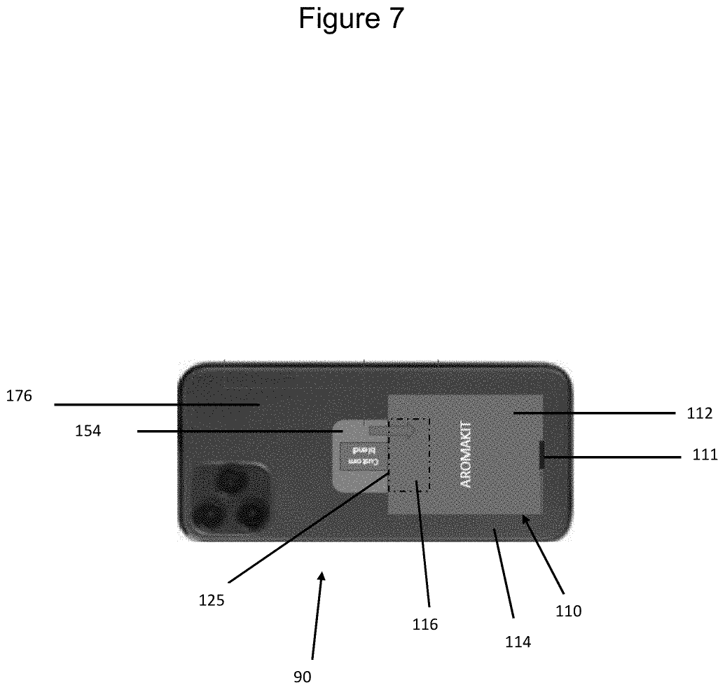

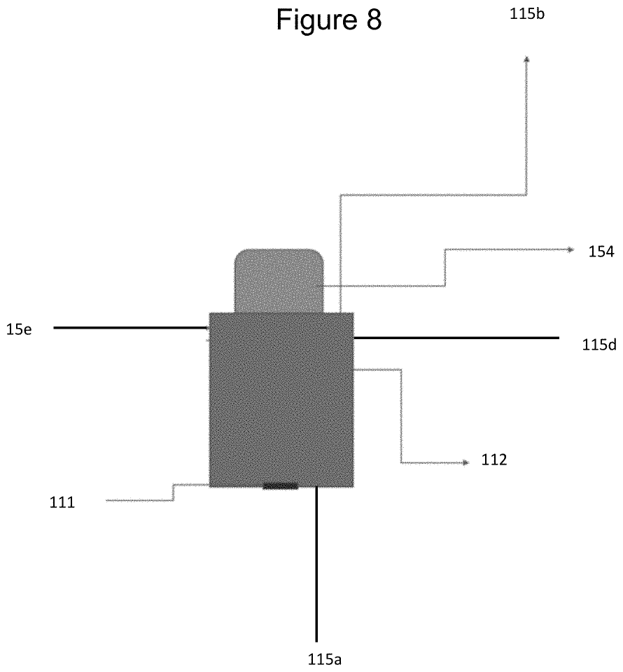

Referring to FIGS. 7 and 8, an exemplary second embodiment of an aromatherapy system 110 is shown when mounted to a smartphone or other computer to form an assembly 92. In this embodiment, the tablet containing scented material is inserted in a slot 125 formed in the side wall of the body 112 that is opposite to the opening 111. The system 110 includes a body 112 configured to be removably mounted to a computer 114. In the illustrated embodiment, the computer 114 is a smartphone. The body 116 includes at least one cavity 116 that is configured to receive a scented component which can be, for example, an aromatherapy tablet 154 including, for example, an oil-soaked pad that will deliver aromatherapy to the user through the scented component 118 which is heated.

Referring to FIGS. 7 and 8, some embodiments of the present disclosure are directed to a system 110 for providing aromatherapy to a user. In some embodiments, the system 110 includes a diffuser 20 similar to that shown in the embodiment of FIG. 1. In some embodiments, the diffuser 20 includes the body 112. In some embodiments, the body 112 is sufficiently sized to hold all the components of the system 110. In some embodiments, the body 112 includes more than one cavity 116. In some embodiments, the cavities 116 are accessible via a top of the body, a side of the body (as shown in the figure), a bottom of the body, a back of the body, a front of the body, or combinations thereof. In some embodiments, the diffuser 20 includes one or more heating elements 30 similar to that shown in the embodiment of FIG. 1. In some embodiments, the heating elements 30 are configured to provide heat to the cavities 116. In some embodiments, a plurality of heating elements 30 are provided to allow local application of heat to the cavities 116, e.g., heat may be applied directly to a first section of a cavity but not directly an adjacent second section. In some embodiments, heating elements 30 are positioned on one or more sides of the cavity 116. In some embodiments, the heating elements 30 include an electronic circuit board 40, similar to that of the embodiment of FIG. 1. In some embodiments, the electronic circuit board 40 includes one or more electronic heating rings 42, or other suitably-shaped apparatus. In some embodiments, the cavity 116 is configured to receive the scented component, such as an aromatherapy tablet 154, that includes the scented material. In some embodiments, the scented material is one or more essential oils.

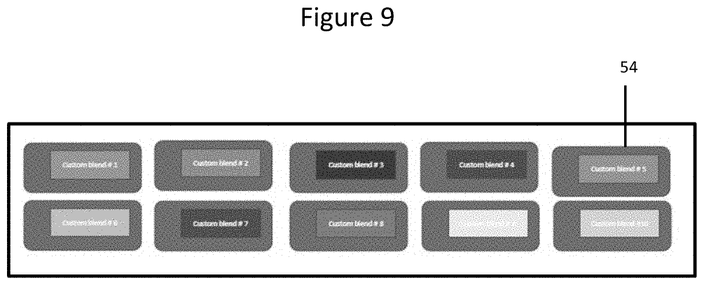

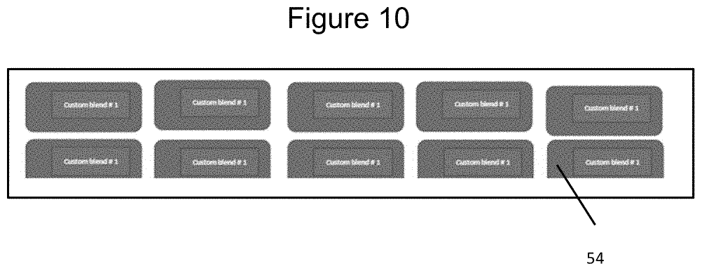

Referring now to FIGS. 9 and 10, in some embodiments, the scented material 50 is provided in the form of aromatherapy tablets 54 formed of or integrated with the scented material 50. In some embodiments, the scented material 50 includes a carrier 52, e.g., a porous pouch enclosing material, a polymeric foam soaked with a liquid material such as an oil. FIG. 9 shows two empty slots 53.

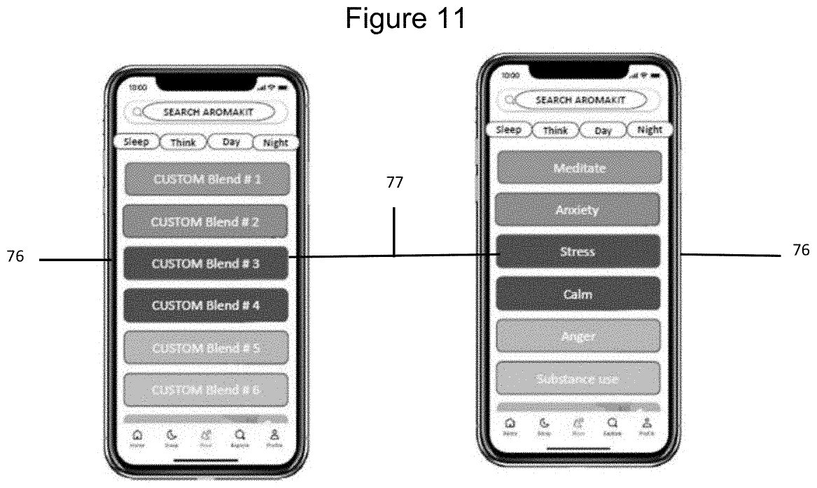

Referring to FIG. 11, in some embodiments, the system includes an aromatherapy module configured to provide a predefined aromatherapy experience to a user. In some embodiments, the predetermined aromatherapy experiences provide scents from the diffuser at predetermined concentrations, durations, combinations, etc., to achieve a desired scent profile in the environment surrounding the system and/or as delivered to a user. In some embodiments, the predetermined aromatherapy experiences are configured to provide particular profiles of scents from the diffuser. In some embodiments, the predetermined aromatherapy experiences are configured to support a user for the purpose of meditation, anxiety reduction, stress reduction, calming, anger reduction, substance use treatment, or combinations thereof. In some embodiments, a user can search and select a predetermined aromatherapy experience from a list provided to them by the system, e.g., via mobile application, as will be discussed in greater detail below.

In some embodiments, the system includes at least one processor 60. In some embodiments, the system includes one or more non-transitory computer storage mediums 66. The non-transitory computer storage medium 66 is encoded with one or more computer programs that, when executed by the processor, control one or more system modules and facilitate actions by those modules and/or the system. In some embodiments, the system includes a control module configured to activate and deactivate the heating elements, and thus control the rate at which the scent material is activated and scent released.

In some embodiments, the system includes one or more communication modules. In some embodiments, the system is in communication with one or more mobile and/or remote computing devices, e.g., a mobile phone, tablet computer, laptop computer, desktop computer, smartwatch, etc. In some embodiments, the communication modules are responsible for sending communications to and receiving communications from the remote computing devices. In some embodiments, the system is in communication with the remote computing devices via a wired connections, wireless connection, or combinations thereof. In some embodiments, the wireless connection includes Wi-Fi, Bluetooth®, near-field communication, cellular network, and the like, or combinations thereof. Referring now to FIG. 3, in some embodiments, the system is controlled via a system mobile application. In some embodiments, the system mobile application is integrated into a third-party application, e.g., as an additional service provided within the third-party application itself. In some embodiments, the system mobile application coordinates with or is controlled by one or more third-party applications, e.g., a third-party exercise application might integrate a predetermined aromatherapy experience into an exercise routine and send instructions to the system mobile application to coordinate delivery of the experience at the appropriate time during the user's exercise. In an exemplary embodiment, a user sends control instructions to the system via a software application executed on the remote computing device. In some embodiments, the software application is stored locally on the remote computing device. In some embodiments, the software application is stored remotely and accessed through a wired or wireless connection.

To use the aromatherapy system 10, a user sends control instructions to the system via one or more software applications 70 executed on the processor 60. In some embodiments, the software application is stored locally on a remote computing device 76. In some embodiments, the software application 70 is stored remotely and accessed through a wired or wireless connection 72. The control instructions are received and implemented by the relevant aromatherapy modules 74. In some embodiments, the user searches for a desired predetermined aromatherapy experience, e.g., one or more known scents or blends of scents, for stress reduction, for anger management, etc. In some embodiments, the user instructs the system to provide the predetermined aromatherapy experience. In some embodiments, the predetermined aromatherapy experience instructions are identified by the aromatherapy module 74, which then informs the control module 78 to provide specific heating instructions to the heating elements 30 to activate the scented material 50 at predetermined intervals, e.g., to deliver the predetermined aromatherapy experiences. In some embodiments, the control module 78 is configured to provide alerts to the user, e.g., to take breaks, and/or to provide aromatherapy experiences at predetermined times of the day or for certain durations, etc. In some embodiments, as the aromatherapy experience progresses, status data, e.g., experience progress, scent material levels, etc., are reported back to the user.

In some embodiments, the system includes one or more power sources 80. The power source 180 is configured to provide the power necessary for the function of the system elements discussed above. In some embodiments, the power source includes sufficient power reserves to enable a plurality of aromatherapy experiences. In some embodiments, the system is wired to a remote power source. In some embodiments, the power source is integrated with the system, e.g., the body of the diffuser 20. In some embodiments, the power source includes one or more batteries. In some embodiments, the power source can be charged via a power cord, e.g., USB type-C. In some embodiments, the system is charged via a computing device to which it is attached, e.g., as seen in FIG. 1. In some embodiments, the power source can be charged wirelessly. In some embodiments, the power source is charged only when its energy level is low. As will be understood by those of skill in the art, the overall battery life of the system depends at least in part in the usage pattern of the system by the user.

In some embodiments, the system includes one or more securing mechanisms 86. In some embodiments, the securing mechanisms are configured to secure the system to a surface. In some embodiments, the surface is any location where the user desires an aromatherapy experience to be delivered, e.g., a wall, a surface on a piece of furniture, e.g., a desk, table, etc., the back of a mobile device, and the like. In some embodiments, the securing mechanism includes adhesive, hook-and-loop fasteners, magnets, etc., or combinations thereof. In some embodiments, the system is integrated directly into a case for a mobile device, e.g., a protective mobile phone case.

Methods and systems of the present disclosure advantageously allow for user to have control over their aromatherapy experiences. Interchangeable scented materials are easily inserted for use and replaced when exhausted. The system is lightweight and can be positioned on the same mobile phone that the user is operating to control it. As users typically have their mobile phone on or around them, this means the system is also on or around the user at almost all times, and thus the delivery of aromatherapy experiences is streamlined for delivery of the therapeutic benefits of essential oils during day-to-day activities and at moments when they would have the most impact, e.g., a stress reducing therapy can be administered during a unforeseen stressful period in the user's work day. The scented materials can be provided and activated in predetermined combinations and durations to achieve a desired result, e.g., stress-relief, anger management, meditation, etc.

In some embodiments, the device can be utilized with different mobile devices. Non-limiting examples include mobile cellular devices or mobile tablets.

In some embodiments, the shape of the case may include on or more geometric designs, e.g. a square, a rectangle, a circle, and the like.

In some embodiments, the scented material can come in other forms. In some embodiments, as presented in FIG. 8, the scented material would come. In the form of an aromatherapy tablet with a liquid oil. In some embodiments, the aromatherapy tablet would instead be a pod, with a powdered scented material, instead of an oil, which would dissolve as the device is used and the scent is released.

In some embodiments, the user can adjust the strength of the scented material released. As seen in FIG. 6, the user can choose to adjust the strength of the released scent according to a low, medium, and high control. In some embodiments, the user can also adjust a timer, to allow the release of the scented material for a specific length of time.

These are variations that fall within the scope of the invention.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

Example 1: An aromatherapy system configured to be connected to a computer was made using the methods described above. More specifically, a case (a body) was fabricated having a first enclosure configured to support a heat sink and a printed circuit board. The heat sink was made from 1060 aluminum, and had a length of 20 mm, a width of 15 mm and a thickness of 6 mm. The heat sink was mounted to a 22 ohm resistor and LM35 heating unit with a thermal adhesive that provides an efficient means for the resistor to transfer as much of its energy as possible into the heat sink, and subsequently into an oil-soaked pad. In this example, the pad was formed from cellulose and had a length of 19 mm, a width of 14 mm and a thickness of 5 mm. The printed circuit board (PCB) was designed using open source software (Fritzing). The finished circuit board had a length of 50 mm, a width of 43 mm. A case dimensioned to house the PCB and heat sink was formed from carbon reinforced nylon, which is suitable for use at the temperature to which the oil is to be heated. The PCB and heat sink were placed in the case and connected to a smartphone using a micro USB cable. The heat sink was heated to 50 degrees, it is noted that when the heat sink was heated to 50 Deg. C, the bottom exterior surface of the case was still at room temperature, which means that higher temperatures also can be used without damaging the smartphone.

What is claimed is:

1. A system for providing aromatherapy to a user, the system comprising:
a mobile computing device including a mobile application to control the system;
a diffuser mounted to the mobile computing device, the diffuser including:
a body;
one or more cavities in the body;
one or more heating elements positioned to provide localized heating to the one or more cavities;
a processor; and
one or more non-transitory computer storage mediums,
a plurality of carriers including scented material, the carriers sized and configured to reversibly fill a cavity and position scented material proximate a heating element,
wherein the non-transitory computer storage mediums are encoded with one or more computer programs that, when executed by the processor in response to instructions from the mobile computing device, instruct the heating elements to apply heat from the heating elements to activate the scented material and deliver a predetermined aromatherapy experience to the user.

2. The system according to claim 1, wherein the predetermined aromatherapy experience includes at least one of meditation, anxiety reduction, stress reduction, calming, anger reduction, and substance use treatment.

3. The system of claim 1, wherein the mobile computing device comprises a smartphone.

4. The system of claim 1, wherein the body is configured to be removably attached to the smartphone.

5. The system of claim 1, wherein the computing device can be heated by a micro USB,

6. The system of claim 1, wherein the computing device may operate wirelessly.

7. The system of claim 1, further comprising of a battery which is charged through a micro USB wire.

8. The system of claim 1, further comprising of the battery of claim 7, which is wirelessly charged.

9. The system of claim 1, wherein the system connects to the mobile computing device through a wireless connection.

10. The system of claim 1, wherein the system connects to the mobile computing device through a Bluetooth connection.

11. The system of claim 1, wherein the carriers comprise scented pads.

12. The system of claim 1, wherein the scented material includes a scented liquid.

13. The system of claim 12, wherein the scented liquid comprises an oil.

14. The system of claim 1, wherein the carriers comprise absorbent pads containing scented liquid.

15. A method comprising:
obtaining a system for providing aromatherapy to a user, the system compromising of a body, one or more cavities in the body, one or more heating elements positioned to provide localized heating the one or more cavities, a processor, and one or more non-transitory computer storage mediums,
inserting a scented component in at least one cavity of the body, and
activating the heating element using a computing device to diffuse scented material.

16. The method of claim 15, further comprising:
activating the heating element through a Micro USB wire connected to the mobile computing device.

17. The method of claim 15, wherein the heating element is connected to the mobile computing device through a wireless connection.

18. The method of claim 15, wherein the heating element is connected to the mobile computing device through a Bluetooth connection.

19. The method of claim 15, wherein the scented component includes a carrier and a scented liquid.

20. The method of claim 19, wherein the scented liquid comprises an oil.
